(19) United States
(12) Patent Application Publication
(10) Pub. No.: US 2026/0345642 A1
(43) Pub. Date: Nov. 5, 2026

(54) METHODS, COMPOSITIONS, AND FORMULATIONS FOR ALLEVIATING CANNABINOID OVERDOSE SYMPTOMS

(71) Applicant: DuggerIPLLC, Albuquerque, NM (US)

(72) Inventor: Vance Dugger, Albuquerque, NM (US)

(73) Assignee: DuggerIPLLC, Albuquerque, NM (US)

(21) Appl. No.: 19/650,858

(22) Filed: Apr. 17, 2026

Related U.S. Application Data
(60) Provisional application No. 63/791,319, filed on Apr. 18, 2025.

(51) Int. Cl. A61K 31/00; A61K 9/20; A61K 31/198; A61K 31/455; A61K 36/53; A61K 36/67; A61P 1/08; A61P 9/06; A61P 25/22

(52) U.S. Cl. CPC A61K 31/658; A61K 9/2013; A61K 9/2054; A61K 31/198; A61K 31/455; A61K 36/53; A61K 36/67; A61P 1/08; A61P 9/06; A61P 25/22

(57) ABSTRACT

Described herein are compositions, formulations, and methods for treating acute tetrahydrocannabinol (THC) overdose or toxicity in a subject, including an adult, child, or animal. Compositions, formulations, and methods described herein comprise use of cannabidiol (CBD) isolate, L-theanine, alpha-lipoic acid, or combinations thereof for reversing or alleviating the symptoms of a THC overdose. The compositions may further include additional ingredients that improve the experience of the subject or further provide symptom alleviating benefits, including botanicals, herbs, flavoring agents, electrolytes, anti-caking agents, binding agents, etc. The subject may experience relief from the THC overdose symptoms within about 5 to 20 minutes upon consumption of the THC overdose composition described herein.

### RELATED APPLICATION

The present Application for patent claims the benefit of U.S. Provisional Patent Application No. 63/791,319 entitled “METHODS, COMPOSITIONS, AND FORMULATIONS FOR ALLEVIATING CANNABINOID OVERDOSE SYMPTOMS,” filed on Apr. 18, 2025, assigned to the assignee hereof, and expressly incorporated herein.

### FIELD

The following is related to methods, compositions, and formulations for alleviating adverse effects associated with tetrahydrocannabinol (THC) overconsumption and cannabinoid toxicity. More specifically, the invention relates to nano-emulsified tablets comprising cannabidiol (CBD) and complementary active ingredients, designed to provide rapid symptomatic relief through enhanced bioavailability and targeted physiological intervention for treating or alleviating the symptoms experienced with cannabinoid toxicity and overdose.

### BACKGROUND

Marijuana is the one of the most commonly used illicit drugs in the United States and is highly used all over the world for both recreational and medicinal use. Derived from the Cannabis plant, marijuana can be consumed by smoking or eating foods that include the marijuana. Tetrahydrocannabinol (THC) is a naturally occurring compound found in the cannabis plant and is a psychoactive compound responsible for the “high” sensation associated with cannabis. Binding to cannabinoid 1 (CB1) receptors in the brain, THC may lead to alterations in mood, perception, and cognitive function.

Marijuana consumption is becoming more common as jurisdictions around the world decriminalize and, in some locations, legalize the use of marijuana medicinally and/or recreationally. The increasing legalization and commercial availability of cannabis products has led to a corresponding rise in cases of acute THC toxicity and cannabinoid overconsumption, particularly among inexperienced users, pediatric populations, and individuals with low cannabinoid tolerance. THC toxicity and overconsumption can lead to acute adverse effects, including but not limited to: temporary feelings of paranoia, panic, fear, and/or anxiety; dizziness; shortness of breath; pupil dilation; vomiting and/or nausea; fast heart rate; increased blood pressure; seizures; shaking that is hard to control; feeling cold; dry mouth; pale skin color; unresponsiveness; disorientation or hallucinations; and hangover.

Additionally, THC can be difficult to dose particularly when differing marijuana products include differing potencies of THC. Smoking or consuming “edibles,” which in some cases have delayed effects, may also lead a marijuana user to consume more than they otherwise would have. For example, an unintended user may unknowingly consume marijuana, such as a child or animal.

Current clinical management of THC overdose remains largely unexplored, with no FDA-approved pharmaceutical intervention specifically targeting the underlying mechanisms of THC-induced toxicity. The lack of therapeutic approaches providing fast-acting symptomatic relief of THC-induced toxicity creates significant patient distress and burden on emergency medical services. Therefore, there is a long-felt but unsolved need for methods to effectively alleviate acute adverse effects of THC overconsumption and cannabinoid toxicity.

Embodiments of the present invention solve this problem by providing methods, compositions, and formulations for products that relieve the symptoms of THC overdose or overconsumption.

### SUMMARY

Provided herein are compositions, formulations, and methods of use for alleviating one or more adverse effects associated with THC overconsumption and cannabinoid-related intolerance events., In some embodiments, the method comprise administering to a subject in need thereof a composition, comprising cannabidiol (CBD) together with one or more complementary active ingredients selected from L-theanine, gamma-aminobutyric acid (GABA), magnesium glycinate, black pepper extract, lemon balm extract, niacinamide (vitamin B3), active terpenes, derivatives thereof, or combinations thereof. In embodiments, the composition may further comprise botanicals or natural medicinal or experience enhancing components (e.g., organic dried lemon powder), sweeteners (e.g., pure cane sugar), sodium (e.g., from pink Himalayan sea salt), binding agents (e.g., tapioca maltodextrin, gum Arabic), anti-caking agents (e.g., silicon dioxide), flavoring agents, coloring agents, excipients, and combinations thereof. In some embodiments, one or more of the ingredients of the composition, are nano-emulsified to increase bioavailability and/or speed up absorption and efficacy. In some embodiments, the formulation further comprises one or more pharmaceutically acceptable excipients to support blending, compression, taste, and uniform dosing.

In some embodiments, the actives are combined with nano-infusion components to form a nanoemulsion that is subsequently blended with excipients and compressed into tablets. In preferred embodiments, the nanoemulsified tablets enhance dispersion and promote more rapid onset relative to conventional liquid or solid formulations. In some embodiments, the tablets are formulated as sublingual tablets to further support faster delivery and user convenience during acute events.

Provided herein are methods of use, including administering an effective amount of the nano-infused sublingual tablet to a subject experiencing known or suspected adverse effects associated with THC overconsumption, thereby helping to reduce subjective distress and support return to comfort. The disclosed compositions and processes provide a standardized, scalable approach for producing consistently dosed, rapid-use tablets intended for symptomatic relief in cannabinoid overconsumption scenarios.

In an aspect of the present disclosure, the composition comprises between approximately 5 mg and approximately 500 mg of CBD. In an aspect of the present disclosure, the composition comprises between approximately 100 mg and approximately 1,000 mg of L-theanine. In an aspect of the present disclosure, the composition comprises between approximately 5 mg and approximately 500 mg of black pepper extract. In an aspect of the present disclosure, the composition comprises between approximately 20 mg and approximately 2,000 mg of magnesium glycinate. In an aspect of the present disclosure, the composition comprises between approximately 50 mg and approximately 2,000 mg of GABA. In an aspect of the present disclosure, the composition comprises between approximately 50 mg and approximately 2,000 mg of lemon balm extract. In an aspect of the present disclosure, the composition comprises between approximately 5 mg and approximately 500 mg of niacinamide.

In an aspect, the present disclosure provides a method of alleviating an adverse THC event comprising administering an effective amount of a composition comprising cannabidiol (CBD) together with one or more complementary active ingredients selected from L-theanine, gamma-aminobutyric acid (GABA), magnesium glycinate, black pepper extract, lemon balm extract, niacinamide (vitamin B3), active terpenes, to a subject to lower the subject's heart rate and alleviate other effects of the THC overdose, where the subject is experiencing a known or suspected acute THC overdose.

In an aspect of the present disclosure, the effective amount of the composition comprises between approximately 5 mg and approximately 500 mg of CBD. In some embodiments, the effective amount of the composition comprises approximately between 50 mg GABA to 2,000 mg GABA. In some embodiments, the effective amount of the composition comprises approximately between 20 mg magnesium glycinate to 2,000 mg magnesium glycinate. In some embodiments, the effective amount of the composition comprises approximately between 1 mg black pepper extract to 200 mg black pepper extract. In some embodiments, the effective amount of the composition comprises approximately between 25 mg lemon balm extract to 2,000 mg lemon balm extract. In some embodiments, the effective amount of the composition comprises approximately between 1 mg niacinamide to 500 mg niacinamide. In some embodiments, the effective amount of the composition comprises approximately between 0.1 mg active terpenes to 50 mg active terpenes. In an aspect of the present disclosure, the composition further comprises organic dried lemon powder, pure cane sugar, pink Himalayan sea salt, one or more natural binding agents selected from the group comprising tapioca maltodextrin, and gum Arabic, one or more natural anti-caking agents selected from the group comprising silicon dioxide, flavoring agents, or combinations thereof.

Embodiments of the present disclosure provide several benefits, including the formulation, manufacturing, and usage of a THC overdose or overconsumption antidote (or relief) composition. The composition of the present disclosure provides for rapid absorption as one or more of the CBD isolate and complementary active ingredients may be nanoemulsified, ensuring a quick onset of effects. In some embodiments, the composition of the present disclosure aids in anxiety relief as L-theanine promotes relaxation and reduces anxiety. In some embodiments, the composition of the present disclosure aids in anxiety relief as gamma-aminobutyric acid (GABA) promotes relaxation and reduces anxiety. In some embodiments, the composition of the present disclosure may provide stress support and relaxation due at least in part to magnesium glycinate included in the composition. In some embodiments, the composition of the present disclosure may provide improved bioavailability and functional support due at least in part to black pepper extract included in the composition. In some embodiments, the composition of the present disclosure aids in calming support due at least in part to lemon balm extract included in the composition. In some embodiments, the composition of the present disclosure may provide metabolic and cellular support due at least in part to niacinamide (vitamin B3) included in the composition. In some embodiments, the composition of the present disclosure further provides flavor and functional support due at least in part to active terpenes included in the composition. The composition formulations disclosed herein provide a comprehensive, fast-acting remedy for THC overconsumption, leveraging nanotechnology and scientifically supported ingredients for optimal results. The composition formulations disclosed herein provide a comprehensive, fast-acting remedy for THC overconsumption, leveraging nanotechnology and scientifically supported ingredients for optimal results.

While specific embodiments and applications have been illustrated and described, the present disclosure is not limited to the precise methods, compositions, and formulations described herein. Various modifications, changes, and variations which will be apparent to those skilled in the art may be made in the arrangement, operation, and details of the compositions, methods and systems disclosed herein without departing from the spirit and scope of the overall disclosure.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

FIG. 1 illustrates a flowchart illustrating a method for administering a Tetrahydrocannabinol (THC) overdose or overconsumption antidote (or relief) composition to a subject, according to aspects of the present disclosure.

FIG. 2 illustrates a flowchart illustrating a method of manufacturing a THC overdose or overconsumption antidote (or relief) composition according to aspects of the present disclosure.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the below prevails unless otherwise stated.

Embodiments of the invention provide compositions, formulations, and methods to alleviate the symptoms of a temporary overdose or overconsumption of a cannabinoid substance, such as tetrahydrocannabinol (THC). Particularly, a THC overdose or overconsumption antidote (or relief) composition of the present disclosure includes cannabidiol (CBD), including CBD isolate, to alleviate one or more adverse effects associated with THC overconsumption. Without being bound by theory, CBD may modulate cannabinoid signaling and may reduce anxiety-like effects and other discomforts associated with high-THC exposure through interaction with non-intoxicating pathways (e.g., serotonergic and other neuromodulatory systems) and/or by functional modulation of CB1-mediated effects. In some embodiments, CBD is incorporated into an oil phase (e.g., MCT oil) and combined with a nano-infusion component (e.g., Nano-S) to form a stabilized nano-dispersion, which may increase dispersion in saliva and improve consistency of absorption upon sublingual administration.

In preferred embodiments, the infusion components comprise MCT oil and “Nano-S.” As used herein, Nano-S refers to a powdered nano-dispersion and encapsulation agent configured to associate with and encapsulate oil-phase constituents, thereby converting the oil-phase constituents into a population of nanoscale particles dispersible in an aqueous environment. As used herein, Nano-S reduces effective droplet/particle size and stabilizes the resulting dispersion to promote faster and more consistent absorption of the oil-based ingredients. Nano-S is a micro-granular amorphous carrier comprising positively charged nano-sized pores and a high internal porosity. In embodiments, Nano-S functions as a substantially inert carrier with minimum reactivity toward cannabis extracts and other active ingredients. The internal pore structure provides high specific surface area and pore volume, which can support formation of solid dispersions and/or stabilization of active ingredients in an amorphous state within the carrier pores. In some embodiments, Nano-S is configured to adsorb oil-phase constituents into its pore network by capillary forces, thereby converting an oil phase (e.g., comprising MCT oil and one or more oil-associated actives) into a free-flowing powder or powder-like intermediate suitable for blending and compression. In embodiments, Nano-S exhibits high oil adsorption capacity (e.g., up to about 3.3 mL oil per gram of Nano-S) while retaining free-flowing properties, thereby reducing oil leakage during downstream processing and supporting scalable tableting.

Without being bound by theory, when oil-associated actives are adsorbed within Nano-S pores as a loaded solid dispersion and subsequently contacted with saliva during sublingual administration, the actives may be released from the pore network by diffusion and/or desorption, presenting an increased effective surface area and supporting rapid dispersion in the oral cavity. In some embodiments, use of Nano-S in preparing the nano-emulsified and/or nano-dispersed formulation improves in-use dispersion and/or dissolution behavior of one or more oil-associated active ingredients relative to an otherwise comparable formulation not incorporating Nano-S. Without being bound by theory, Nano-S may support such performance by adsorbing oil-phase constituents into a high-porosity pore network, increasing effective surface area, and facilitating release of the active ingredients by diffusion and/or desorption upon contact with aqueous fluids. In preferred embodiments, the use of Nano-S in preparing the nano-emulsified and/or nano-dispersed formulation enhances bioavailability of one or more oil-associated active ingredients (e.g., CBD and one or more terpenes) relative to an otherwise comparable formulation not incorporating Nano-S. Without being bound by theory, Nano-S may enhance bioavailability by reducing effective particle and/or droplet size of oil-phase constituents, increasing effective surface area upon contact with saliva, improving dispersion stability in an aqueous environment, and promoting more consistent mucosal and/or gastrointestinal absorption.

Compositions of the present disclosure may include L-theanine (y-glutamylethylamide; theanine; N5-ethyl-L-glutamine), an amino acid that may promote relaxation and attenuate anxiety and panic symptoms associated with acute THC overconsumption. Without being bound by theory, L-theanine may modulate excitatory/inhibitory neurotransmission (e.g., by influencing glutamatergic signaling and supporting inhibitory tone) and thereby reduce subjective distress, restlessness, and agitation that can accompany cannabinoid overconsumption.

Compositions of the present disclosure may further comprise one or more complementary active ingredients selected from: gamma-aminobutyric acid (GABA), magnesium glycinate, black pepper extract, lemon balm extract, niacinamide (vitamin B3), and active terpenes. These actives may be included to target distinct symptom domains commonly reported during cannabinoid overconsumption events.

For example, compositions of the present disclosure may include GABA, which may support inhibitory neurotransmission and promote a calming effect. Without being bound by theory, GABA may help reduce acute anxiousness, restlessness, and tension associated with THC overconsumption. Compositions of the present disclosure may include magnesium glycinate, a bioavailable magnesium source that may support neuromuscular relaxation and normal autonomic function. Without being bound by theory, magnesium supplementation may assist in reducing jitteriness, muscle tension, and subjective “overstimulation” sometimes reported during high-THC experiences.

Compositions of the present disclosure may include lemon balm extract (Melissa officinalis). Without being bound by theory, lemon balm extract may provide calming effects and support relaxation, including by modulating neurotransmitter systems associated with stress responses, thereby complementing CBD and L-theanine. Compositions of the present disclosure may include black pepper extract (e.g., comprising piperine and/or pepper-derived constituents). Without being bound by theory, black pepper extract may provide functional support for cannabinoid intolerance events, including by modulating sensory perception and/or influencing metabolic and transport pathways that affect the onset and intensity of orally delivered actives.

Compositions of the present disclosure may include niacinamide (vitamin B3) to support cellular energy metabolism and physiologic recovery during an acute adverse cannabinoid event. Without being bound by theory, niacinamide may support NAD+-dependent pathways involved in cellular stress response and may improve overall tolerability.

Compositions of the present disclosure may include active terpenes and/or terpene-containing botanicals. In some embodiments, the composition includes lemon-derived components (e.g., lemon powder) providing terpenes such as limonene. Without being bound by theory, terpenes may contribute to symptom modulation (e.g., reduction of perceived intensity of intoxication, improved mood, and/or reduced anxiety), and may also improve flavor and patient compliance.

In embodiments, the foregoing active ingredients may be combined in a compressed sublingual tablet formulated for rapid disintegration/dissolution in the mouth. In some embodiments, one or more actives are incorporated into a nano-infused mixture to yield a stabilized dispersion upon contact with saliva, thereby providing more rapid and consistent delivery. In embodiments, L-theanine, alpha-lipoic acid, GABA, magnesium glycinate, lemon balm extract, black pepper extract, niacinamide, terpenes, or combinations thereof, may enhance the effectiveness of CBD in alleviating symptoms associated with temporary THC overdose or overconsumption by providing multi-pathway support for anxiety/panic, agitation, nausea, and general physiologic discomfort.

As used herein, the term “composition” refers to a combination of one or more ingredients disclosed herein for treating or alleviating the symptoms experienced with cannabinoid toxicity and temporary overdose. “Composition” as referred to herein may otherwise be referred to as a THC overdose or overconsumption antidote (or relief) composition, an antidote composition, a formulation, or the like.

As used herein, the term “formulation” refers to a composition of the present disclosed prepared for a particular purpose, such as liquid dosing, solid dosing, etc.

As used herein, the term “THC overdose” refers to a subject experiencing one or more of the following: temporary feelings of paranoia, panic, fear, and/or anxiety; dizziness; shortness of breath; pupil dilation; vomiting and/or nausea; fast heart rate; increased blood pressure; seizures; shaking that is hard to control; feeling cold; dry mouth; pale skin color; unresponsiveness; disorientation or hallucinations; and hangover. At the time of filing, a “THC overdose” is not defined by any amount of THC in the bloodstream of a subject, or by any other quantifier. That is, “THC overdose” as used herein is subjective to the subject experiencing the overdose symptoms or to an observer. “THC overdose” may otherwise be referred to as poisoning, toxicity, overconsumption, acute overdose, temporary overdose, and the like. It should be understood that “THC overdose” as used herein is not limited by or associated with death.

As used herein, the term “overconsumption” refers to an excessive intake of THC or a THC-comprising product at once or over a period of time resulting in adverse symptoms associated with a THC overdose.

As used herein, the term “antidote” refers to a composition that treats (i.e., counteracts or improves) one or more of the effects associated with THC overconsumption. An “antidote” as used herein may relieve or lessen temporary feelings of paranoia, panic, fear, and/or anxiety; dizziness; shortness of breath; pupil dilation; vomiting and/or nausea; seizures; shaking that is hard to control; dry mouth; unresponsiveness; disorientation or hallucinations; and hangover. An “antidote” as used herein may lower heart rate; lower blood pressure; regulate body temperature; and bring color back to a subject's skin.

As used herein, the term “alleviate” refers to reducing, improving, or entirely eliminating a THC overdose symptom experienced by a subject.

As used herein, the term “adverse event” refers to an undesirable experience associated with the use of marijuana and derivatives thereof by a subject.

As used herein, the term “symptom” refers to a physical or mental disturbance, and may be subjective or objective. Subjective symptoms of a THC overdose may include feelings of paranoia, panic, fear, and/or anxiety; dizziness; pupil dilation; vomiting and/or nausea; seizures; shaking that is hard to control; dry mouth; pale skin color; unresponsiveness; disorientation or hallucinations; and hangover. Objective symptoms of a THC overdose may include shortness of breath; increased heart rate; increased blood pressure; and low body temperature, each of which may be determined by a healthcare professional and may be based on the subject's age, height, weight, etc.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

As used herein, including in the claims, “or” as used in a list of items (e.g., a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an example step that is described as “based on condition A” may be based on both a condition A and a condition B without departing from the scope of the present disclosure. In other words, as used herein, the phrase “based on” shall be construed in the same manner as the phrase “based at least in part on.”

As used herein, “sublingual tablet” refers to a solid, unit-dose, compressed tablet formulation configured for placement under the tongue (i.e., in the sublingual space) where it dissolves and/or disintegrates in saliva to release one or more active ingredients for absorption through the sublingual and/or oral mucosa, with or without a portion being swallowed for gastrointestinal absorption. Sublingual tablets are distinguished from conventional swallowed tablets by being formulated to provide a relatively rapid onset of effect and improved patient convenience during acute-use situations.

As used herein, “sublingual tablet” includes tablets that substantially dissolve/disintegrate in the mouth without chewing and may be characterized as “compressed sublingual tablets” or “melt-in-mouth” tablets. In embodiments, the sublingual tablets described herein are compressed oral tablets manufactured to a predetermined target tablet weight and include excipients selected to facilitate compression, mechanical integrity, organoleptic properties, and controlled disintegration/dissolution in the mouth, including one or more sugars and/or sugar alcohols, directly compressible diluents, binders, glidants and/or lubricants, flavors, and colors. In some embodiments, the sublingual tablet comprises a nano-infused mixture of oil-associated active ingredients combined with an excipient blend and compressed to form the unit-dose tablet, such that upon sublingual administration the actives are released as a stabilized dispersion to support relatively rapid and consistent uptake.

In some embodiments, Nano-S provides improved flowability and compressibility of blends comprising oil-associated actives due to its internal granular structure and high porosity. In embodiments, this can improve tabletability and mechanical integrity of compressed sublingual tablets and can facilitate manufacture of hard tablets with reduced risk of oil weeping or leakage during compression and handling.

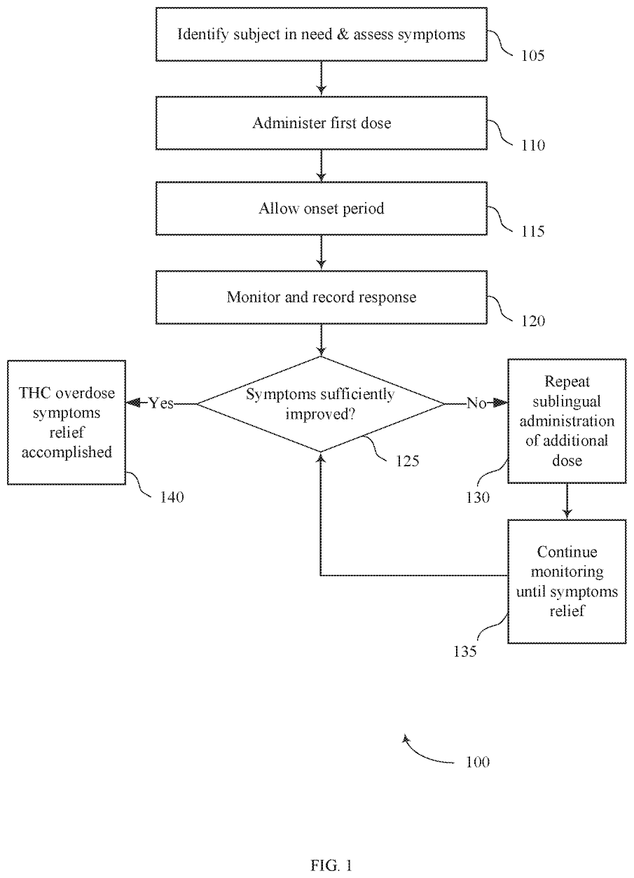

FIG. 1 illustrates an example method for administering the THC overdose antidote (or relief) composition disclosed herein to a subject, in accordance with the present disclosure. The method may include identifying that a marijuana consumer (e.g., an adult, a child, an animal) is experiencing cannabinoid toxicity or temporary overdose symptoms (e.g., temporary feelings of paranoia, panic, fear, and/or anxiety; dizziness; shortness of breath; pupil dilation; vomiting and/or nausea; fast heart rate; increased blood pressure; seizures; shaking that is hard to control; feeling cold; dry mouth; pale skin color; unresponsiveness; disorientation or hallucinations; and hangover). The identification may be made by any observer (e.g., a friend, parent, healthcare professional, bystander), including the subject experiencing the overdose. The composition may be administered or consumed by the subject to relieve the symptoms of the THC overdose or overconsumption, according to methods disclosed herein. The subject may experience relief from the adverse symptoms in about 5 to 20 minutes upon consuming the composition.

In some embodiments, compositions and formulations of the present disclosure may include one or more additional ingredients to enhance the efficacy, flavor, mouthfeel, benefits, packaging, storage, etc. of the final composition product. Particularly, compositions and formulations of the present disclosure may include one or more flavor enhancers, electrolytes, binding agents, anti-caking agents, excipients, or combinations thereof.

An exemplary formulation for a THC overdose or overconsumption antidote (or relief) composition capable of, among other things, calming anxiety and lowering a subject's heart rate associated with a temporary overdose of THC, comprises a composition comprising CBD isolate, L-theanine, and alpha-lipoic acid. One or more of the CBD isolate, L-theanine, and alpha-lipoic acid may be nano-emulsified. In one embodiment, CBD isolate, L-theanine, and alpha-lipoic acid, or a combination thereof are the only ingredients of the composition.

CBD isolate refers to CBD that has been isolated from THC. Typically, CBD isolate is in the form of a crystalline, flavorless powder. In embodiments, the CBD isolate is at least about 75% pure, or more particularly at least about 80% pure (i.e., free of compounds other than CBD), or more particularly at least about 85% pure, or more particularly at least about 90% pure, or more particularly at least about 95% pure, or more particularly at least about 99% pure, or more particularly at least about 99.5% pure, or more particularly at least about 99.9% pure. Compatible in powder formulations, water-based formulations, and oil-based formulations, CBD isolate is incorporated into any formulation type disclosed herein. CBD isolate may be procured from any known source, including but not limited to, the hemp plant, the marijuana plant, or synthetically.

Gamma-aminobutyric acid (GABA) refers to an amino acid and inhibitory neurotransmitter found naturally in humans and other organisms. Typically, GABA is provided as a white or off-white powder. In embodiments, GABA is compatible in powder formulations, water-based formulations, and oil-based formulations, and is incorporated into any formulation type disclosed herein. GABA may be procured from any known source, including but not limited to, fermentation-derived sources (e.g., microbial fermentation), plant-derived sources, or synthetically produced sources.

Magnesium glycinate refers to a chelated magnesium compound comprising magnesium bound to glycine. Typically, magnesium glycinate is provided as a white or off-white powder. In embodiments, magnesium glycinate is incorporated into any formulation type disclosed herein, including powder formulations and compressed tablet formulations. Magnesium glycinate may be procured from any known source, including but not limited to, mineral-derived magnesium processed into chelated form, and may be manufactured according to known methods.

Black pepper extract refers to an extract derived from Piper nigrum and may include piperine as an active constituent. Typically, black pepper extract is provided as a powder. In embodiments, black pepper extract is incorporated into any formulation type disclosed herein, including powder formulations and compressed tablet formulations, and may be included for functional support and/or to complement other active ingredients. Black pepper extract may be procured from any known source, including standardized botanical extracts.

Lemon balm extract refers to an extract derived from Melissa officinalis. Typically, lemon balm extract is provided as a powder. In embodiments, lemon balm extract is incorporated into any formulation type disclosed herein, including powder formulations and compressed tablet formulations. Lemon balm extract may be procured from any known source, including standardized botanical extracts.

Niacinamide (vitamin B3) refers to a form of vitamin B3 and is typically provided as a white crystalline powder. In embodiments, niacinamide is incorporated into any formulation type disclosed herein, including powder formulations and compressed tablet formulations. Niacinamide may be procured from any known source, including but not limited to, synthetic sources and/or fermentation-derived sources.

Active terpenes refer to terpene compounds that may be derived from botanical sources and incorporated for flavor and functional support. Typically, active terpenes are provided as oils or concentrates, which may be added at low levels to one or more formulations disclosed herein. In embodiments, active terpenes are incorporated into powder formulations, oil-based formulations, and compressed tablet formulations, including as part of an infusion step. Active terpenes may be procured from any known source, including but not limited to, plant-derived extracts and/or synthetically produced terpene compositions.

L-theanine may be procured from any known source but is generally derived from the leaves of Camellia sinensis plant in any oxidation state (e.g., green tea, oolong tea, black tea). Additionally or alternatively, L-theanine may be sourced from mushrooms. L-theanine may also be manufactured synthetically.

In some embodiments, the composition further comprises Alpha-lipoic acid. Alpha-lipoic acid refers to an organosulfur compound that may provide antioxidant support. Alpha-lipoic acid may be procured from any known source, but is generally derived from animal sources (e.g., cow, pig, lamb, etc.) and particularly organ meats, plant sources (e.g., beets, broccoli, brussels sprouts, peas, potatoes, yams, etc.), or is synthetically manufactured.

In an embodiment, the composition disclosed herein includes one or more botanicals (e.g., plants, fruits, or herbal species) to enhance the efficacy or experience associated with the composition. For example, the one or more botanicals may lower a subject's heart rate and calm anxiety through a synergistic effect. Dried lemon powder, for example, contains limonene terpene which may modulate the effects of THC. The one or more botanicals may also improve the flavor or color, for example, of the composition. The enhancing botanical ingredients may include a whole species, parts of the species, or extracts of the species, such as an oil. The enhancing ingredients may be in the form of an oil, a powder, a suspension, or a liquid. Non-limiting examples of composition enhancing species include lemon, lavender, rosemary, milk thistle, dandelion root, adaptogens, spices such as cinnamon and turmeric, and the like.

In some embodiments, other inactive ingredients may be included in the composition to improve the experience of the subject ingesting the composition, such as flavoring agents, texture enhancing agents, coloring agents, and so forth. The experience enhancing component(s) may be in the form of extracts, volatile oils, powders, or any commercially available form. The experience enhancing component(s) may be derived naturally or artificially (i.e., synthetically), resulting in imitation products, or a combination thereof. The experience enhancing component(s) can be protected with mixed tocopherols.

In a non-limiting example, the experience enhancing component(s) may include a flavor enhancer such as sweetener, such as sugar (e.g., pure cane sugar, or sugar derivative), honey, agave, stevia, monk fruit, and the like. The sweetener may aid in balancing the blood sugar of the subject. Non-limiting examples of other flavoring agents include, but are not limited to, real or imitation strawberry extract or powder, lime extract or powder, fruit punch extract or powder, (pure) vanilla extract or powder, (pure) anise extract, real or imitation banana extract, real or imitation cherry extract, chocolate extract, (pure) lemon extract, (pure) orange extract, (pure) peppermint extract, real or imitation pineapple extract, imitation rum extract, pure or imitation cocoa or cacao extract or powder, (pure) cinnamon powder, extract or oil, (pure) cardamom powder, extract or oil, (pure) nutmeg powder, extract or oil, (pure) clove powder, extract or oil, or volatile oils, such as balm oil, bay oil or bergamots oil, cedar-wood oil, cherry oil, walnut oil, cinnamon oil, clove oil, or peppermint oil, and so forth.

In another example, the experience enhancing component(s) may include salt (e.g., sea salt, table salt, kosher salt), electrolytes (e.g., sodium, chlorine, magnesium, calcium, potassium), or a combination thereof which may aid at least in improving hydration, and combating dry mouth.

In another example, the experience enhancing component(s) may include coloring agents such as, but not limited to, naturally derived dyes from turmeric, saffron, elderberry, spirulina, hibiscus, carotenoids (e.g., carrots, paprika), betanin (e.g., beets), chlorophyllin, spinach, matcha, etc., and/or synthetically derived agents such as azorubine, allura red, sunset yellow, tartrazine, brilliant blue, indigo carmine, etc. Other coloring agents are readily apparent to the skilled artisan and selection of suitable coloring agents may depend, in part, on the formulation of the composition.

In some embodiments, other inactive ingredients may be included to aid in the formulation of the composition, such as binding agents (or binders), anti-caking agents, etc. Binding agents may aid in holding the ingredients of the composition formulation together, and in some cases improve the mouth feel of the composition. For example, some binding agents may create a melt-in-mouth texture. Non-limiting examples of binding agents may include tapioca maltodextrin, gum Arabic, guar gum, xanthan gum, flax seed, chia seeds, pectin, starch, psyllium, agar, egg, carrageenan, gelatin, etc. Anti-caking agents, like the name suggests, may prevent the composition formulation from clumping together typically by absorbing moisture and may aid in ease of packaging, transport, flowability, and consumption. The anti-caking agents may be used to prevent clumping of the final composition product with others of the composition product and/or to prevent clumping during manufacturing.

Non-limiting examples of anti-caking agents include silicon dioxide, calcium silicate, magnesium stearate, magnesium carbonate, tricalcium phosphate, starch, etc. Binding and anti-caking agents included in the composition may be derived naturally or artificially. Other binding and/or anti-caking agents not expressly listed herein are readily apparent to the skilled artisan and selection of suitable binding and/or anti-caking agents may depend, in part, on the formulation of the composition.

In some embodiments of the present invention, the THC temporary overdose alleviating composition further comprises one or more excipients. The excipients are inactive ingredients that can be used as carriers for the active ingredients of the formulation. Examples of excipients include, but are not limited to, water (e.g., filtered water, distilled water, mineral water, alkaline water), oils (e.g., sweet almond oil, coconut oil, olive oil, rose oil), pullulan, chitosan, MCT, dicalcium phosphate, microcrystalline cellulose, stearic acid, croscarmellose sodium, magnesium trisillicate, magnesium stearate, hydroxypropyl methylcellulose, ethyl cellulose, hypromellose, and so forth. Other excipients are readily apparent to the skilled artisan and selection of suitable excipients may depend, in part, on the formulation of the composition.

In some embodiments of the present invention, emulsifiers are added for stability of the composition. Examples of emulsifiers include, but are not limited to, lecithin (e.g., from egg or soy), and/or mono- and di-glycerides. Other emulsifiers are readily apparent to the skilled artisan and selection of suitable emulsifiers may depend, in part, on the formulation of the composition.

The components of the composition disclosed here may be derived naturally or artificially (e.g., synthetically). That is, one or more of the products may be derived naturally, and one or more of the components may be derived artificially. In embodiments, one or more of the components of the composition may be organic. In embodiments, one or more of the components of the composition may be non-organic.

In accordance with embodiments of the present disclosure, a method for providing relief from a temporary or acute THC overdose or overconsumption, comprises administering the composition in an appropriate dosage. The dosage may be based on weight, height, sex, age, overdose severity, formulation of the composition (e.g., liquid versus solid, versus gel, etc.), amount of THC or marijuana consumed, hours since THC/marijuana consumption, etc. Further, the THC overdose composition may be administered at least once during a predetermined time interval, for example, per hour, per day, per week, and so forth. While there may be a minimum dose that is expected to relieve the THC overdose symptoms of an individual, consuming more or less than the minimum dose will not cause harm to the individual. That is, the composition may be formulated so that the person experiencing the THC overdose symptoms and who may not be able to determine the correct dose on their own, may be able to take an amount of the composition without inducing further side effects or toxicity.

Formulations, such as a dose (or unit), of the present disclosure may comprise between about 5 mg and about 500 mg of CBD isolate, or more preferably between about 10 mg and about 400 mg of CBD isolate, or more preferably between about 15 mg and about 300 mg of CBD isolate, or even more preferably between about 20 mg and about 200 mg of CBD isolate.

FIG. 1 illustrates a flowchart illustrating a method for administering a Tetrahydrocannabinol (THC) overdose or overconsumption antidote (or relief) composition to a subject, according to aspects of the present disclosure. The method may include, at 105, identifying that a marijuana consumer (e.g., an adult, a child, an animal) is experiencing cannabinoid toxicity or temporary overdose symptoms (e.g., temporary feelings of paranoia, panic, fear, and/or anxiety; dizziness; shortness of breath; pupil dilation; vomiting and/or nausea; fast heart rate; increased blood pressure; seizures; shaking that is hard to control; feeling cold; dry mouth; pale skin color;

unresponsiveness; disorientation or hallucinations; and hangover). The method may further include, at 110, administering a first dose by placing a tablet sublingually and allowing the tablet to disintegrate or dissolve without chewing. The method may further include, at 115, allowing an onset period by waiting a predefined interval (e.g., about 5-30 minutes; in some embodiments about 5-20 minutes). The method may further include, at 120, monitoring and recording a response by tracking one or more endpoints (e.g., a subject-reported anxiety or panic score, heart rate, and/or nausea). The method may further include, at 125, determining whether symptoms are sufficiently improved and whether the marijuana consumer is eligible for repeat dosing. The method may further include, at 130, permitting a second unit dose and repeating sublingual administration of nanoemulsified tablets when symptoms persist after a set interval (e.g., about 10-60 minutes; in some embodiments about 10-30 minutes). The method may further include, at 135, continuing monitoring until symptoms relief to accomplish THC overdose symptoms relief at 140.

In some embodiments, a method of alleviating one or more adverse effects associated with tetrahydrocannabinol (THC) overconsumption in a subject includes administering to the subject an effective amount of a composition comprising cannabidiol (CBD) and one or more complementary active ingredients, when the subject is experiencing a known or suspected acute THC overconsumption event, thereby alleviating one or more symptoms selected from anxiety, panic, paranoia, tachycardia, elevated blood pressure, nausea, vomiting, dizziness, disorientation, hallucinations, tremor, dry mouth, and combinations thereof.

In some embodiments, the effective amount comprises about 5 mg to about 500 mg of CBD.

In some embodiments, the effective amount comprises about 100 mg to about 1,000 mg of L-theanine. In some embodiments, the effective amount comprises about 100 mg to about 1,000 mg of magnesium glycinate. In some embodiments, the effective amount comprises about 100 mg to about 1,000 mg of gamma-aminobutyric acid (GABA). In some embodiments, the effective amount comprises about 100 mg to about 1,000 mg of lemon balm extract. In some embodiments, the effective amount comprises about 20 mg to about 1,000 mg of niacinamide.

In some embodiments, the method further comprises monitoring one or more symptoms at one or more time points after administration.

In some embodiments, the method further comprises administering a second effective amount of the composition if the one or more symptoms persist after about 10 minutes to about 60 minutes.

In some embodiments, the effective amount of the composition comprises approximately 5 mg CBD. In some embodiments, the effective amount of the composition comprises approximately 20 mg CBD. In some embodiments, the effective amount of the composition comprises approximately 40 mg CBD. In some embodiments, the effective amount of the composition comprises approximately 60 mg CBD. In some embodiments, the effective amount of the composition comprises approximately 80 mg CBD. In some embodiments, the effective amount of the composition comprises approximately 100 mg CBD. In some embodiments, the effective amount of the composition comprises approximately 120 mg CBD. In some embodiments, the effective amount of the composition comprises approximately 140 mg CBD. In some embodiments, the effective amount of the composition comprises approximately 160 mg CBD. In some embodiments, the effective amount of the composition comprises approximately 180 mg CBD. In some embodiments, the effective amount of the composition comprises approximately 200 mg CBD. In some embodiments, the effective amount of the composition comprises approximately 220 mg CBD. In some embodiments, the effective amount of the composition comprises approximately 240 mg CBD. In some embodiments, the effective amount of the composition comprises approximately 260 mg CBD. In some embodiments, the effective amount of the composition comprises approximately 280 mg CBD. In some embodiments, the effective amount of the composition comprises approximately 300 mg CBD. In some embodiments, the effective amount of the composition comprises approximately 320 mg CBD. In some embodiments, the effective amount of the composition comprises approximately 340 mg CBD. In some embodiments, the effective amount of the composition comprises approximately 360 mg CBD. In some embodiments, the effective amount of the composition comprises approximately 380 mg CBD. In some embodiments, the effective amount of the composition comprises approximately 400 mg CBD. In some embodiments, the effective amount of the composition comprises approximately 420 mg CBD. In some embodiments, the effective amount of the composition comprises approximately 440 mg CBD. In some embodiments, the effective amount of the composition comprises approximately 460 mg CBD. In some embodiments, the effective amount of the composition comprises approximately 480 mg CBD. In some embodiments, the effective amount of the composition comprises approximately 500 mg CBD.

In some embodiments, the effective amount of the composition comprises approximately 5 mg CBD. In some embodiments, the effective amount of the composition comprises approximately 25 mg CBD. In some embodiments, the effective amount of the composition comprises approximately 45 mg CBD. In some embodiments, the effective amount of the composition comprises approximately 65 mg CBD. In some embodiments, the effective amount of the composition comprises approximately 85 mg CBD. In some embodiments, the effective amount of the composition comprises approximately 105 mg CBD. In some embodiments, the effective amount of the composition comprises approximately 125 mg CBD. In some embodiments, the effective amount of the composition comprises approximately 145 mg CBD. In some embodiments, the effective amount of the composition comprises approximately 165 mg CBD. In some embodiments, the effective amount of the composition comprises approximately 185 mg CBD. In some embodiments, the effective amount of the composition comprises approximately 205 mg CBD. In some embodiments, the effective amount of the composition comprises approximately 225 mg CBD. In some embodiments, the effective amount of the composition comprises approximately 245 mg CBD. In some embodiments, the effective amount of the composition comprises approximately 265 mg CBD. In some embodiments, the effective amount of the composition comprises approximately 285 mg CBD. In some embodiments, the effective amount of the composition comprises approximately 305 mg CBD. In some embodiments, the effective amount of the composition comprises approximately 325 mg CBD. In some embodiments, the effective amount of the composition comprises approximately 345 mg CBD. In some embodiments, the effective amount of the composition comprises approximately 365 mg CBD. In some embodiments, the effective amount of the composition comprises approximately 385 mg CBD. In some embodiments, the effective amount of the composition comprises approximately 405 mg CBD. In some embodiments, the effective amount of the composition comprises approximately 425 mg CBD. In some embodiments, the effective amount of the composition comprises approximately 445 mg CBD. In some embodiments, the effective amount of the composition comprises approximately 465 mg CBD. In some embodiments, the effective amount of the composition comprises approximately 485 mg CBD.

The amount of the other inactive ingredients per unit of composition may be selected based on the total number of unit servings of composition administered to a subject. Moreover, the total amount of the other ingredients may also be selected, in part, based on the health condition of the subject.

In one embodiment, the methods of the present disclosure utilize the compositions of the present disclosure for administration to a subject with a diagnosis of a temporary overdose or overconsumption of THC. A subject may feel relief or easing of the overdose symptoms in about 5 to 20 minutes upon consumption of the composition disclosed herein. The time to experience the effects of the composition may be based on weight, height, sex, age, overdose severity, formulation of the composition (e.g., liquid versus solid, liquid versus gel, etc.), amount of THC or marijuana consumed, hours since THC/marijuana consumption, etc. For example, effects may be felt within 10 to about 15 minutes for a dry tablet composition formulation. In another example, effects may be felt within 5 to about 10 minutes for a liquid composition formulation.

In embodiments, compositions of the present disclosure may be sold over the counter (without a prescription or advice from a healthcare professional), and/or may be prescribed and/or administered by a healthcare professional.

In some embodiments, a method of alleviating one or more adverse effects associated with THC overconsumption in a subject comprises administering to the subject an effective amount of a composition comprising cannabidiol (CBD), wherein the subject is experiencing a known or suspected acute THC overconsumption event.

In some embodiments, administering the composition alleviates one or more symptoms selected from anxiety, panic, paranoia, tachycardia, elevated blood pressure, nausea, vomiting, dizziness, disorientation, hallucinations, tremor, dry mouth, and combinations thereof.

In some embodiments, the composition is administered sublingually.

In some embodiments, the composition is provided as a compressed sublingual tablet configured to disintegrate and/or dissolve in the mouth.

In some embodiments, the effective amount comprises about 5 mg to about 500 mg of CBD.

In some embodiments, at least a portion of the CBD is nano-emulsified or nano-dispersed.

In some embodiments, the CBD is incorporated into an oil phase comprising MCT oil and combined with a nano-dispersion and encapsulation agent to form a nano-infused mixture.

In some embodiments, the effective amount comprises about 100 mg to about 1,000 mg of L-theanine.

In some embodiments, the composition further comprises alpha-lipoic acid.

In some embodiments, the effective amount comprises about 100 mg to about 2,000 mg of alpha-lipoic acid.

In some embodiments, the composition further comprises one or more complementary active ingredients selected from gamma-aminobutyric acid (GABA), magnesium glycinate, lemon balm extract, black pepper extract, niacinamide, one or more terpenes, and combinations thereof.

In some embodiments, the subject experiences a reduction in a subject-reported anxiety and/or panic severity score within about 5 minutes to about 30 minutes after administration.

In some embodiments, the subject experiences a reduction in heart rate within about 5 minutes to about 30 minutes after administration.

In some embodiments, the method further comprises monitoring one or more symptoms at one or more timepoints after administration.

In some embodiments, the method further comprises administering a second effective amount of the composition if at least one symptom persists after about 10 minutes to about 60 minutes following administration of a first effective amount.

In some embodiments, the composition is administered in a unit-dose format.

In some embodiments, the subject is a pediatric subject.

In some embodiments, the subject is an animal.

In some embodiments, the composition is administered during an acute THC overconsumption event caused by ingestion of an edible cannabis product.

In some embodiments, administration is performed at a baseline timepoint after the subject reports acute distress associated with THC exposure, and symptom severity is recorded at baseline and at one or more post-administration timepoints comprising about 5 minutes, about 10 minutes, about 15 minutes, about 20 minutes, and about 30 minutes.

In some embodiments, at least one endpoint comprises a subject-reported anxiety and/or panic severity score.

In some embodiments, at least one endpoint comprises heart rate.

In some embodiments, a responder is defined as a subject achieving a predetermined reduction in at least one symptom severity score within a predetermined time period after administration.

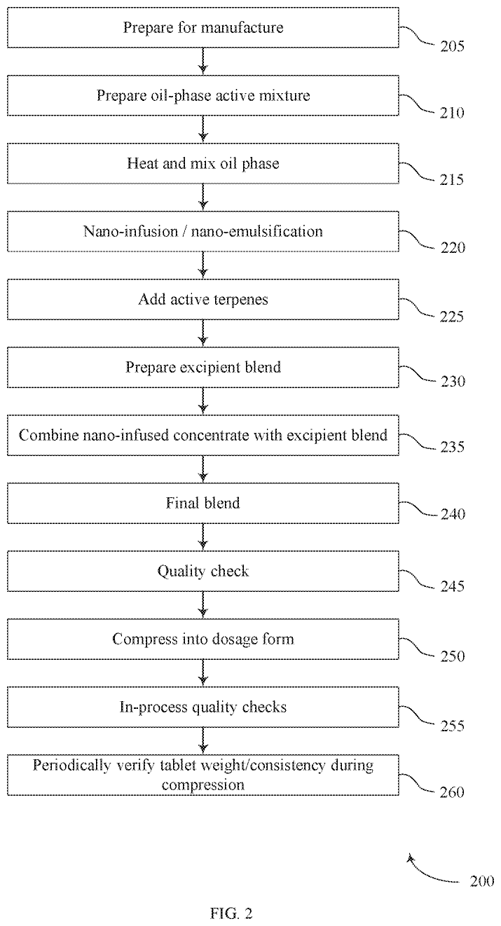

Methods of the present disclosure are directed to manufacturing the THC overdose composition, such as the method depicted in FIG. 2. FIG. 2 illustrates a flowchart illustrating a method of manufacturing a THC overdose or overconsumption antidote (or relief) composition according to aspects of the present disclosure. The method may include, at 205, calibrating equipment and preparing for manufacture by gathering and weighing components. The method may further include, at 210, preparing an oil-phase active mixture by combining CBD and other active ingredients with MCT oil to form an oil phase (in some embodiments, at about a 1:1 mass ratio of MCT to active mass). The method may further include, at 215, heating and mixing the oil phase (in some embodiments, at about 75 °C for about 15-20 minutes) until the oil phase is uniform. The method may further include, at 220, performing nano-infusion or nano-emulsification by adding a nano-emulsifying or nano-dispersion agent (e.g., Nano-S) and high-shear mixing or immersion blending until a uniform nano-infused concentrate forms. The method may further include, at 225, adding active terpenes and mixing until uniform. The method may further include, at 230, combining excipients by preparing an excipient blend and pre-blending to uniformity (in some embodiments, using a V-mixer for about 15 minutes). The method may further include, at 235, combining the nano-infused concentrate with the excipient blend by slowly introducing the concentrate into the excipients during mixing to reduce lumping and improve uniform distribution. The method may further include, at 240, performing a final blend by blending to uniformity (in some embodiments, using a V-mixer for about 20 minutes) to form a tableting blend. The method may further include, at 245, performing a quality check by sampling and verifying uniform distribution. The method may further include, at 250, compressing into a dosage form by feeding the tableting blend into a tablet press, setting a target tablet weight, performing a test press, and adjusting settings as needed. The method may further include, at 255, performing in-process quality checks. The method may further include, at 260, periodically verifying tablet weight and consistency during compression.

The composition of the present disclosure may be formulated into a single dose format or a multi-dose format. For example, the composition may be formulated so a subject need only consume a single dose. In another example, the THC overdose composition may be formulated so that a subject consumes multiple doses over a period of time. A single dose format may be beneficial for ease of administration or consumption by a subject and a multi-dose formulation may be beneficial for slowing raising blood sugar, lowering heart rate, etc.

In some embodiments, the composition may be formulated in a one-part format or a multi-part format. A one-part format may comprise all the ingredients of the composition formulated into a single consumable format. A multi-part format may separate one or more of the ingredients into different compositions. The different compositions may be combined prior to consumption by a subject, upon consumption by a subject, or may be consumed at different times. For example, one or more of the composition ingredients are administered in a first formulation and the remaining ingredients are administered in a second formulation, where the first and second formulations may be combined prior to consumption, consumed separately but at the same time, or consumed separately at different times. A one-part formulation may be beneficial for ease of storage, consumption by a subject, among other things. A multi-part formulation may be beneficial in maintaining freshness and/or reducing reactions between ingredients of the composition.

In embodiments, the composition may be formulated into sublinguals, pills, tablets, caplets, capsules, powders, suspensions, gels or liquids, and vapors, optionally comprising an acceptable carrier such as but not limited to water, milk, juice, sodas, starch, vegetable oils, salt solutions, hydroxymethyl cellulose, carbohydrates, and so forth. The composition may be formulated for oral consumption, nasal consumption, or via injection. In one embodiment of the present disclosure, a composition is formulated with or into foods or liquids. Examples of foods or liquids include, but are not limited to, a single serving bar, “candy”, gummy, chocolate, or beverage. In one embodiment of the present disclosure, the ingredients or the composition is dried and made readily soluble in water and other consumable liquids as described herein.

Compositions of the present disclosure may be stored in any method known to maintain freshness, efficacy, aid in transport and storage, provide ease of use to a consumer, etc. For example, compositions of the present disclosure may be stored in airtight containers, blister packs, foil, bottles, syringes (oral or injectable), etc. and is based on the formulation of the composition (i.e., liquid formulation, solid formulation, multi-part or multi-dose formulation, etc.).

In the example illustrated in FIG. 2, a manufacturer may gather the ingredients and equipment needed. The ingredients may be any of those disclosed herein, including CBD and complementary active ingredients, including gamma-aminobutyric acid (GABA), magnesium glycinate, black pepper extract, lemon balm extract, niacinamide (vitamin B3), and active terpenes. In some embodiments, the ingredients further include recipient (excipient) ingredients suitable for compressed sublingual tablets, including one or more sugars and/or sugar alcohols, microcrystalline cellulose, magnesium stearate, mannitol, flavoring agents (including lemon powder), and colorants, and combinations thereof. Equipment may include a calibrated analytical scale, a heat-safe infusion vessel, an oven capable of about 75 °C, a high-shear mixer or immersion blender, a V-mixer, a tablet press, PPE, and packaging materials (e.g., bottles, blister packs, or airtight containers).

In some embodiments, an active ingredient nano-infusion is prepared by combining the active ingredients in a heat-safe vessel and mixing until uniform. In some embodiments, a carrier oil (e.g., MCT oil) is added at about a 1:1 ratio to the active ingredient mass, and the mixture is heated at about 75 °C for about 15-20 minutes. The mixture may then be removed from the oven. In some embodiments, a nano-infusion component (e.g., “Nano S”) is added at about a 1:1 ratio to the CBD+carrier oil mass and blended using high-shear mixing or an immersion blender until fully infused. In some embodiments, active terpenes are added and mixed thoroughly. In some embodiments, addition of Nano-S to the warmed oil phase comprising MCT oil and one or more oil-associated active ingredients results in adsorption of the oil phase into Nano-S pores to form a free-flowing, non-tacky, powder-like nano-infused intermediate. This intermediate can be readily metered and blended with excipients to improve distribution uniformity and to support compression into tablets with reduced oil leakage In some embodiments, the Nano-S addition ratio is selected to provide sufficient pore capacity to adsorb the oil phase and maintain a free-flowing intermediate suitable for downstream blending and compression. In embodiments, the Nano-S addition ratio may be selected based at least in part on the amount and composition of the oil phase (e.g., MCT oil content and the mass of oil-associated active ingredients) and a target flowability of the resulting Nano-S loaded intermediate. In further embodiments, the Nano-S addition ratio is selected to reduce or prevent oil weeping or leakage during mixing, storage, and tablet compression.

In some embodiments, the excipient ingredients are measured and mixed to ensure even distribution. In some embodiments, the recipient ingredients are combined and pre-blended in a V-mixer for about 15 minutes.

In some embodiments, the nano-infused active mixture is combined with the recipient blend based on the final formulation of the composition. In some embodiments, the nano-infused active mixture is slowly reintroduced into the recipient blend and mixed in a V-mixer for about 20 minutes. In some embodiments, blend uniformity is verified visually and/or by sampling.

In an example of a final compressed sublingual tablet formulation, the blended material is transferred to a tablet press and compressed to a target tablet weight (e.g., about 1.70 g). In some embodiments, a test press is performed and tablet weight is verified prior to a full compression run. In some embodiments, in-process quality checks are performed at a defined interval (e.g., every 30 tablets) to verify tablet weight. Upon completion, the tablets may be packaged in airtight containers or blister packs. Labels may be placed on the containers or packs that provide information regarding ingredients, dosage instructions, disclaimers (e.g., “Consult a healthcare professional prior to use”), contraindications, and the like. The product may be stored in a cool, dark location.

It should be well understood that the examples disclosed herein, such as those described with reference to FIGS. 1 and 2 and merely exemplary embodiments. The concepts illustratively disclosed suitably may be practiced in the absence of any element which is not specifically disclosed herein. It is apparent to those skilled in the art that many changes, variations, modifications, other uses, and applications of the disclosure are possible, and changes, variations, modifications, other uses, and applications which do not depart from the spirit and scope of the disclosure are deemed to be covered by the disclosure. Thus, various embodiments may omit, substitute, or add various procedures or components as appropriate. For instance, it should be appreciated that the methods may be performed in an order different than that described, and that various steps may be added, omitted or combined. Also, aspects and elements described with respect to certain embodiments may be combined in various other embodiments. It should also be appreciated that the following composition and methods may individually or collectively be components of a larger composition or system, wherein other procedures may take precedence over or otherwise modify their application. The foregoing discussion has been presented for purposes of illustration and description.

Having described various features of this disclosure both generally and specifically in the preceding detailed description, the following exemplification provides a specific example of the preparation of the subject matter described herein. By way of this example, and in the context of the preceding detailed description, the skilled person will immediately recognize variations to the method set forth in the example. The following exemplification is illustrative only and shall not limit this disclosure or any patent claim that matures from this disclosure. Any patent claim that matures from this disclosure shall instead be limited by the explicit features recited in the claim in the context of its claim dependency and according to conventional principles of claim construction as applied in view of this disclosure.

### EXEMPLIFICATION

### Example 1. Preparation of a Nano-Emulsified Sublingual CBD Tablet for Alleviating Symptoms Associated with THC Overconsumption

This Example describes preparation of a compressed sublingual nano-emulsified tablet comprising cannabidiol (CBD) and complementary active ingredients, formulated for rapid symptomatic relief of adverse effects associated with tetrahydrocannabinol (THC) overconsumption, cannabinoid toxicity, and temporary cannabinoid overdose.

The dosage form is a compressed sublingual tablet with a target unit mass of approximately 1.70 g (1700 mg).

TABLE 1
Excipients (recipient ingredients)

| Ingredient | Batch amount (g) |
|---|---|
| Castor sugar | 103.0 |
| Fructose DC | 90.0 |
| Dextrose DC | 90.0 |
| Sorbitol DC | 90.0 |
| Microcrystalline cellulose | 5.0 |
| Magnesium stearate | 5.0 |
| Mannitol | 3.0 |
| Flavor | 30.0 |
| Lemon powder | 20.0 |
| Color | 10.0 |
| Total excipients | 446.0 |

TABLE 2
Active ingredients

| Active ingredient | Batch amount (g) |
|---|---|
| L-theanine | 10.0 |
| Black pepper extract | 0.5 |
| Cannabidiol (CBD) | 2.5 |
| Magnesium glycinate | 10.0 |
| GABA | 10.0 |
| Lemon balm extract | 10.0 |
| Niacinamide (vitamin B3) | 2.0 |
| Active terpenes (optional) | 0.5 |

TABLE 3
Nano-infusion components

| | | Batch |
|---|---|---|
| | | amount |
| Component | Ratio basis | (g) |
| Active ingredients | Reference mass | 45.0 |
| (excluding terpenes) | | |
| MCT oil | 1:1 relative to active | 45.0 |
| | ingredients mass | |
| Nano S | 1:1 relative to | 47.5 |
| (nano-emulsifying system) | (CBD + MCT) mass | |

TABLE 4
Batch mass and expected yield

| Parameter | Value |
|---|---|
| Total excipients mass (g) | 446.0 |
| Total active ingredients mass, excluding terpenes (g) | 45.0 |
| MCT oil mass (g) | 45.0 |
| Nano S mass (g) | 47.5 |
| Optional terpenes mass (g) | 0.5 |
| Total batch mass, including optional terpenes (g) | 584.0 |
| Total batch mass, excluding optional terpenes (g) | 583.5 |
| Target tablet weight (g) | 1.70 |
| Expected yield (tablets) | ~343 |

Equipment cleanliness and calibration were verified and component identities and weights were confirmed using a calibrated analytical scale. The active ingredients were combined in a heat-safe vessel and MCT oil was added at an approximately 1:1 mass ratio relative to the active ingredient mass. The mixture was mixed until uniform and heated at approximately 75 °C for about 15-20 minutes. The mixture was removed from heat and Nano S was added at approximately a 1:1 mass ratio relative to the combined CBD plus MCT mass. The mixture was blended using a high-shear mixer or immersion blender until a uniform nano-infused concentrate was obtained. Optional active terpenes were then added and mixed until uniform.

The excipients were combined separately and pre-blended in a V-mixer for about 15 minutes to form a uniform excipient blend. The nano-infused concentrate was then slowly introduced into the excipient blend while mixing, followed by blending in a V-mixer for about 20 minutes to produce a final uniform blend. The final blend was transferred to a tablet press and compressed to a target tablet mass of about 1.70 g. A test press was performed to verify tablet weight and compression settings, followed by full compression. In-process checks were performed periodically during compression to verify tablet weight consistency.

TABLE 5
Approximate per-tablet active dosing

| | Total batch | Approx. per-tablet |
|---|---|---|
| Active ingredient | amount (mg) | amount (mg) |
| L-theanine | 10,000 | ~29 |
| Black pepper extract | 500 | ~1.5 |
| CBD | 2,500 | ~7.3 |
| Magnesium glycinate | 10,000 | ~29 |
| GABA | 10,000 | ~29 |
| Lemon balm extract | 10,000 | ~29 |
| Niacinamide (vitamin B3) | 2,000 | ~5.8 |
| Active terpenes (optional) | 500 | ~1.5 |

The per-tablet amounts in Table 5 are calculated directly from the batch gram amounts shown in Tables 1-3 and a yield of ~343 tablets.

### Example 2. Observational Data & Phase I Protocol (Hi Hope Rescue Remedy)

### Example 2A. Observational Study Summary

An observational dataset was collected evaluating the effects of Hi Hope Rescue Remedy in individuals experiencing adverse symptoms associated with THC consumption. Nineteen participants (ages 21-43) were included. All participants reported symptom relief within 15-30 minutes following administration of 2-5 melts depending on body mass.

TABLE 6
Observational dataset (participant-reported outcomes)

| | | | THC | | Onset | | Re- |
|---|---|---|---|---|---|---|---|
| ID | Age | Weight | Method | Dose | (min) | Symptoms | lief |
| JD01 | 21 | 150 | Edible | 2 | 20 | Anxiety | Yes |
| JD02 | 23 | 165 | Vape | 2 | 15 | Dizziness | Yes |
| JD03 | 25 | 180 | Flower | 3 | 20 | Tachycardia | Yes |
| JD04 | 27 | 210 | Edible | 4 | 25 | Nausea | Yes |
| JD05 | 29 | 195 | Concentrate | 3 | 20 | Panic | Yes |
| JD06 | 31 | 220 | Beverage | 4 | 30 | Anxiety | Yes |
| JD07 | 34 | 240 | Edible | 5 | 30 | Paranoia | Yes |
| JD08 | 28 | 175 | Vape | 3 | 15 | Anxiety | Yes |
| JD09 | 26 | 160 | Flower | 2 | 20 | Lightheaded | Yes |
| JD10 | 33 | 205 | Concentrate | 4 | 25 | Panic | Yes |
| JD11 | 35 | 230 | Edible | 5 | 30 | Anxiety | Yes |
| JD12 | 37 | 250 | Beverage | 5 | 30 | Nausea | Yes |
| JD13 | 40 | 260 | Concentrate | 5 | 25 | Panic | Yes |
| JD14 | 22 | 155 | Vape | 2 | 15 | Anxiety | Yes |
| JD15 | 24 | 170 | Flower | 3 | 20 | Tachycardia | Yes |
| JD16 | 30 | 200 | Edible | 4 | 25 | Paranoia | Yes |
| JD17 | 38 | 245 | Beverage | 5 | 30 | Anxiety | Yes |
| JD18 | 43 | 270 | Concentrate | 5 | 30 | Panic | Yes |
| JD19 | 32 | 190 | Vape | 3 | 20 | Dizziness | Yes |

### Example 2B. Clinical Observations

Across all participants, the administration of Hi Hope Rescue Remedy demonstrated consistent onset of symptom mitigation within a 15-30 minute window. Symptoms including anxiety, tachycardia, nausea, and paranoia were reduced. No adverse events were reported. These findings suggest potential efficacy in mitigating THC-induced adverse effects.

### Example 2C. Proposed Phase I Clinical Protocol

The objective of the Phase I study is to evaluate safety, tolerability, and preliminary efficacy of Hi Hope Rescue Remedy in individuals experiencing THC-related adverse effects. The study is designed as an open-label, dose-escalation study with 30-50 participants. The inclusion criteria include adults aged 21-50 who have recently consumed THC and who present with adverse symptoms. The dosing includes an initial dose of 2 melts with escalation to 5 melts based on body weight.

The study endpoints include time to symptom relief, symptom severity reduction measured on a 1-10 scale, and adverse event monitoring. Data are collected using time-stamped symptom logs, vital signs, and participant-reported outcomes. This Phase I study will establish a safety profile and support method-of-use claims.

### Example 3. Dosing and Usage

A subject who has consumed THC and is experiencing one or more adverse symptoms is administered Hi Hope Rescue Remedy as an oral composition, for example melts configured to dissolve in the oral cavity. Administration may be initiated after THC consumption when the subject experiences one or more adverse symptoms including anxiety, dizziness, tachycardia, nausea, panic, paranoia, and/or lightheadedness. The THC consumption preceding administration may include ingestion or inhalation of THC, for example via an edible, a vape, flower, a concentrate, and/or a beverage.

An initial dose may include about 2 melts. The dose may be increased based on body weight or body mass, symptom severity, and/or persistence of symptoms to about 3, about 4, or about 5 melts. The subject may be re-dosed after a time interval of about 10 to about 30 minutes if symptoms persist. Symptom mitigation may occur within about 15 to about 30 minutes following administration. Dosing may be repeated as needed until the adverse symptoms are reduced to a tolerable level.

Clinical dosing may be evaluated in an open-label, dose-escalation Phase I study enrolling about 30 to about 50 participants. Eligible participants may include adults about 21 to about 50 years of age who have recently consumed THC and are experiencing one or more adverse symptoms. Participants may receive an initial dose of about 2 melts, with escalation up to about 5 melts based on body weight or body mass. Efficacy endpoints may include time to symptom relief and reduction in symptom severity measured on a numerical scale, for example a 1-10 scale. Safety endpoints may include monitoring for adverse events. Data collection may include time-stamped symptom logs, vital signs, and participant-reported outcomes.

The concepts illustratively disclosed herein suitably may be practiced in the absence of any element which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

The description set forth herein, in connection with the appended drawings, describes example configurations and does not represent all the examples that may be implemented or that are within the scope of the claims. The term “example” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, known structures and devices are shown in block diagram form in order to avoid obscuring the concepts of the described examples.

The description herein is provided to enable a person having ordinary skill in the art to make or use the disclosure. Various modifications to the disclosure will be apparent to a person having ordinary skill in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1. A composition for use in alleviating one or more adverse effects associated with tetrahydrocannabinol (THC) overconsumption in a subject, the composition comprising: cannabidiol (CBD); and
one or more complementary active ingredients, wherein the composition is formulated as an orally administered dosage form configured to provide rapid onset of effect.

2. The composition of claim 1, wherein the dosage form is a compressed sublingual tablet configured to disintegrate and/or dissolve in a mouth of the subject.

3. The composition of claim 1, wherein at least a portion of the CBD is present in a nano-emulsified and/or nano-dispersed form, and wherein the nano-emulsified and/or nano-dispersed form is produced by contacting the CBD with a nano-emulsifying component during manufacture of the composition.

4. The composition of claim 3, wherein the nano-emulsifying component comprises Nano-S, and wherein contacting the CBD with Nano-S during manufacture enhances bioavailability of the CBD and complementary active ingredients.

5. The composition of claim 1, wherein the CBD is incorporated in an oil phase comprising medium-chain triglyceride (MCT) oil.

6. The composition of claim 1, wherein the one or more complementary active ingredients comprise at least one of L-theanine, gamma-aminobutyric acid (GABA), magnesium glycinate, lemon balm extract, niacinamide, and black pepper extract.

7. The composition of claim 1, further comprising active terpenes.

8. The composition of claim 1, wherein a unit dose of the composition comprises about 25 mg CBD.

9. The composition of claim 1, further comprising one or more sugars and/or sugar alcohols.

10. The composition of claim 1, further comprising microcrystalline cellulose, magnesium stearate, a flavoring component, and a colorant.

11. The composition of claim 2, wherein the composition is configured such that the tablet disintegrates sublingually within about 0.5 minute to about 5 minutes.

12. The composition of claim 1, wherein the composition is configured for unit-dose self-administration with an optional second unit dose administered after about 10 minutes to about 60 minutes if at least one symptom persists.

13. A method of alleviating one or more adverse effects associated with tetrahydrocannabinol (THC) overconsumption in a subject, the method comprising:
administering to the subject an effective amount of a composition comprising cannabidiol (CBD) together with other complementary active ingredients, wherein the subject is experiencing a known or suspected acute THC overconsumption event.

14. The method of claim 13, wherein the administering alleviates one or more symptoms selected from anxiety, panic, paranoia, tachycardia, elevated blood pressure, nausea, vomiting, dizziness, disorientation, hallucinations, tremor, dry mouth, and combinations thereof.

15. The method of claim 13, wherein the composition is administered sublingually as a compressed sublingual tablet configured to disintegrate in the mouth.

16. The method of claim 13, wherein at least a portion of the CBD is nano-emulsified or nano-dispersed.

17. The method of claim 13, wherein the effective amount comprises about 5 mg to about 500 mg of CBD.

18. A method of alleviating acute adverse effects associated with THC overconsumption in a subject, the method comprising: placing a sublingual tablet in a sublingual space of the subject, wherein the sublingual tablet comprises the composition of claim 1, and wherein the subject experiences a reduction in anxiety and/or panic and a reduction in heart rate within about 5 minutes to about 30 minutes after placing the sublingual tablet.

19. A method of manufacturing a tablet composition comprising cannabidiol (CBD), the method comprising:
combining CBD with at least one oil to form an oil phase;
combining the oil phase with a nano-emulsifying component to form a nano-emulsified CBD mixture;
blending the nano-emulsified CBD mixture with a powder excipient blend to form a tableting blend; and
compressing the tableting blend to form compressed sublingual tablets.

20. The method of claim 19, wherein combining CBD with the at least one oil comprises combining CBD with medium-chain triglyceride (MCT) oil to form the oil phase, and wherein combining the oil phase with the nano-emulsifying component comprises contacting the oil phase with Nano-S, wherein an amount of Nano-S is selected to provide sufficient pore capacity to adsorb the oil phase and maintain a free-flowing intermediate suitable for downstream blending and compression.
